(19) United States
(12) Patent Application Publication
(10) Pub. No.: US 2006/0258633 A1
(43) Pub. Date: Nov. 16, 2006

(54) Amorphous tegaserod maleate

(76) Inventors: Santiago Ini, Haifa (IL); Michael Pinchasov, Rishon-Lezion (IL); Tamas Koltai, Netanya (IL); Judith Aronhime, Rehovot (IL)

Correspondence Address: KENYON & KENYON LLP, ONE BROADWAY, NEW YORK, NY, 10004, US

(21) Appl. No.: 11/371,840

(22) Filed: Mar. 8, 2006

Related U.S. Application Data
(60) Provisional application No. 60/659,694, filed on Mar. 8, 2005.
(60) Provisional application No. 60/664,124, filed on Mar. 21, 2005.
(60) Provisional application No. 60/724,514, filed on Oct. 6, 2005.
(60) Provisional application No. 60/758,072, filed on Jan. 10, 2006.
(60) Provisional application No. 60/773,566, filed on Feb. 14, 2006.

(51) Int. Cl. A61K 31/33

(52) U.S. Cl. 514/183

(57) ABSTRACT

Provided are amorphous and purely amorphous tegaserod maleate and processes for the preparation thereof. Also provided are pharmaceutical compositions comprising amorphous or purely amorphous tegaserod maleate and methods for the treatment of irritable bowel syndrome using the same.

### CROSS-REFERENCE TO RELATED APPLICATIONS

This application claims the benefit of provisional application Ser. Nos. 60/659,694, filed Mar. 8, 2005; 60/664,124, filed Mar. 21, 2005; 60/724,514, filed Oct. 6, 2005; 60/758,072, filed Jan. 10, 2006; and, AWAITED, filed Feb. 14, 2006 (Attorney Docket No. 1662/87306), which are incorporated herein by reference.

### FIELD OF THE INVENTION

The invention encompasses tegaserod maleate amorphous form and the preparation thereof.

### BACKGROUND OF THE INVENTION

Tegaserod maleate is an aminoguanidine indole 5HT4 agonist indicated for the treatment of irritable bowel syndrome (IBS). Tegaserod maleate is also known as 3-(5-methoxy-1H-indole-3-ylmethylene)-N-pentylcarbazimidamide hydrogen meleate, and has the following structure:

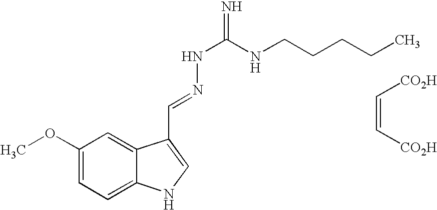

Tegaserod maleate is a white to off-white crystalline powder slightly soluble in ethanol and very slightly soluble in water. Physician's Desk Reference, 57^{th} ed., p. 2339. The marketed polymorphic form of tegaserod maleate (ZELNORM®) is listed in IPCOM000021161D and designated tegaserod maleate Form A. Form A is characterized by an X-ray diffraction pattern with peaks at 5.4, 6.0, 6.6 and 10.8±0.2 degrees two theta. The crystalline form is further characterized by an X-ray diffraction pattern having peaks at about 5.9, 6.4, 11.5, 12.0, 14.8, 15.4, 16.2, 18.1, 19.4, 21.7, 23.9, 26.8 and 29.7±0.2 degrees two theta.

The solid state physical properties of an active pharmaceutical ingredient (API), such as tegaserod maleate, effect the commercial usefulness of the API. Solid state physical properties include, for example, the flowability of the milled solid. Flowability affects the ease with which the material is handled during processing into a pharmaceutical product. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate.

Another important solid state property of a pharmaceutical compound is its rate of dissolution in aqueous fluid. The rate of dissolution of an active ingredient in a patient's stomach fluid may have therapeutic consequences since it imposes an upper limit on the rate at which an orally-administered active ingredient may reach the patient's bloodstream. The rate of dissolution is also a consideration in formulating syrups, elixirs and other liquid medicaments. The solid state form of a compound may also affect its behavior on compaction and its storage stability.

These practical physical characteristics are influenced by the conformation and orientation of molecules in the unit cell, which defines a particular form of a substance. An amorphous form may have thermal behavior different from that of a polymorphic form. A particular form may also give rise to distinct spectroscopic properties that may be detectable by powder X-ray crystallography, solid state C NMR spectrometry and infrared spectrometry. The solid state physical properties of tegaserod maleate may be influenced by controlling the conditions under which it is obtained in solid form.

Tegaserod maleate is disclosed in U.S. Pat. No. 5,510,353 (Example 13) and the equivalent EP 0 505 322. U.S. Pat. No. 5,510,353 (“the '353 patent”) discloses the preparation of tegaserod base by reacting indole-3-carbaldehyde and aminoguanidine in a protic solvent in the presence of inorganic or organic acid (Example 2a describes the reaction in methanol and hydrochloric acid). Tegaserod maleate disclosed in the '353 patent has a melting point of 190° C. (Table 1, Example 13).

The literature (Buchheit K. H, et al., J.Med.Chem., 1995, 38, 2331) describes a general method for the condensation of aminoguanidines with indole-3-carbadehydes in methanol in the presence of HCl (pH 3-4). The product obtained after solvent evaporation may be converted to its hydrochloride salt by treatment of the methanolic solution with diethylether/HCl followed by recrystallization from methanol/diethylether. Tegaserod base prepared according to this general method is characterized solely by a melting point of 155° C. (table 3 compound 5b). Additional Tegaserod maleate characterization was done by ¹H and ¹³C-NMR according to the literature (Jing J. et. al., Guangdong Weiliang Yuansu Kexue, 2002, 9/2, 51).

Chinese patent No. CN 1176077C discloses X-ray diffractograms of two crystalline forms of tegaserod maleate: Form B2 and Form C.

WO 04/085393 discloses four crystalline forms of tegaserod maleate. The search report for WO 04/085393 further identifies WO 00/10526, and Drugs Fut. 1999, 24(1) which provides an overview for tegaserod maleate.

The discovery of new forms of a pharmaceutically useful compound provides an opportunity to improve the performance characteristics of a pharmaceutical product. It enlarges the repertoire of materials that a formulation scientist has available for designing, for example, a pharmaceutical dosage form of a drug with a targeted release profile or other desired characteristic. There is a need in the art for additional processes for preparation of tegaserod maleate amorphous form. Amorphous form often has greater bioavailability than crystalline forms and may be more suitable for formulation of an active pharmaceutical ingredient when greater bioavailability is desired.

### SUMMARY OF THE INVENTION

In one aspect, the invention encompasses amorphous tegaserod maleate. Preferably, the amorphous tegaserod maleate contains less than about 20% crystalline tegaserod maleate by weight, more preferably less than about 10% by weight, and even more preferably less than about 5% by weight.

In another aspect, the invention encompasses purely amorphous tegaserod maleate.

In another aspect, the present invention provides a process of preparing amorphous tegaserod maleate comprising: providing a solution of tegaserod maleate in at least one organic solvent and spray drying the solution to obtain amorphous tegaserod maleate.

In another aspect, the present invention provides a process for preparing amorphous tegaserod maleate comprising: providing a solution of tegaserod maleate and organic solvent and fast-removing the solvent under reduced pressure.

### BRIEF DESCRIPTION OF THE FIGURES

FIG. 1 illustrates an X-ray powder diffraction pattern for amorphous tegaserod maleate according to example 2.

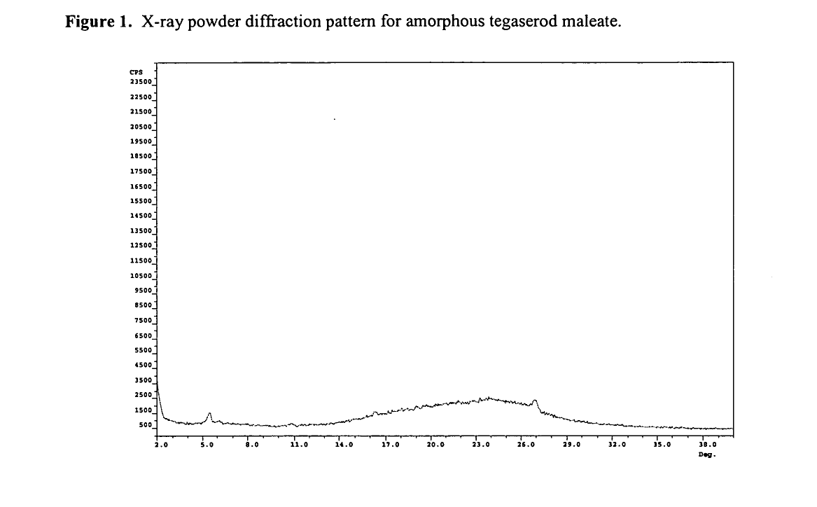

FIG. 2 illustrates an X-ray powder diffraction pattern for purely amorphous tegaserod maleate according to example 4.

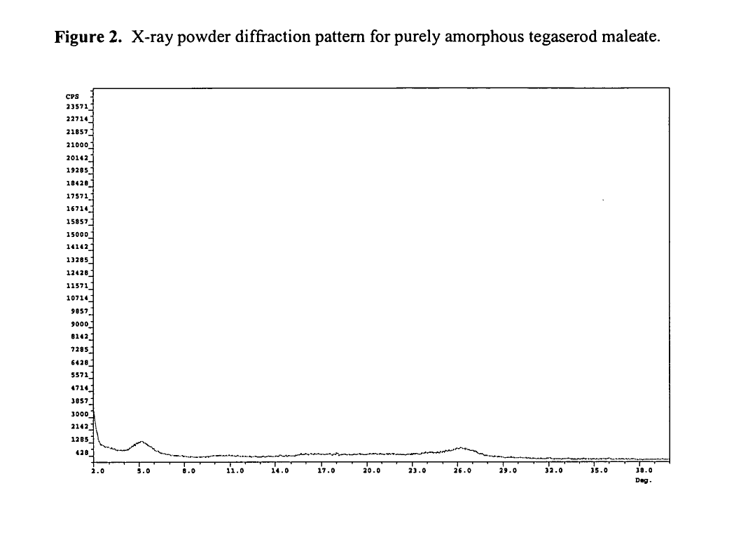

FIG. 3 illustrates an X-ray powder diffraction pattern for amorphous tegaserod maleate according to example 1.

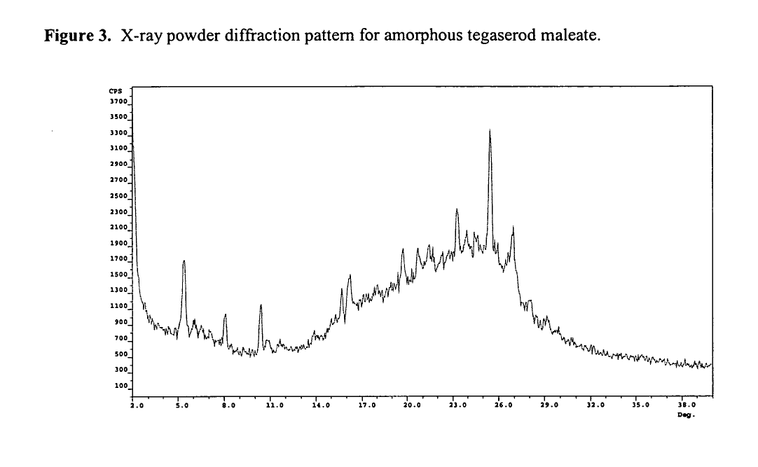

FIG. 4 illustrates an X-ray powder diffraction pattern for amorphous tegaserod maleate according to example 2.

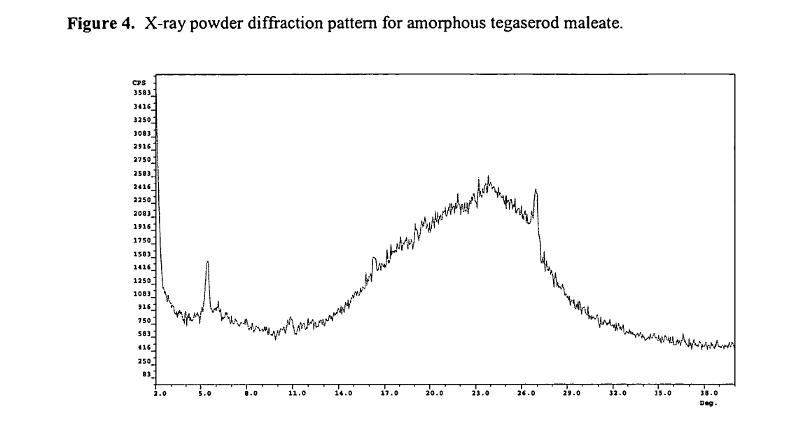

FIG. 5 illustrates an X-ray powder diffraction pattern for amorphous tegaserod maleate according to example 3.

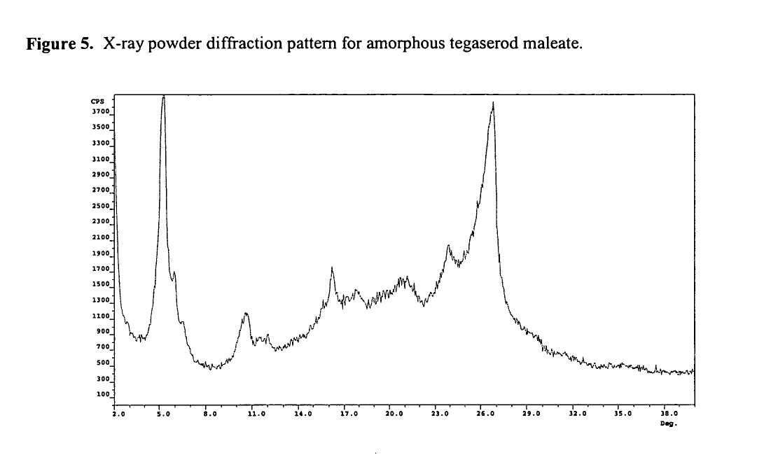

FIG. 6 illustrates an X-ray powder diffraction pattern for purely amorphous tegaserod maleate according to example 5.

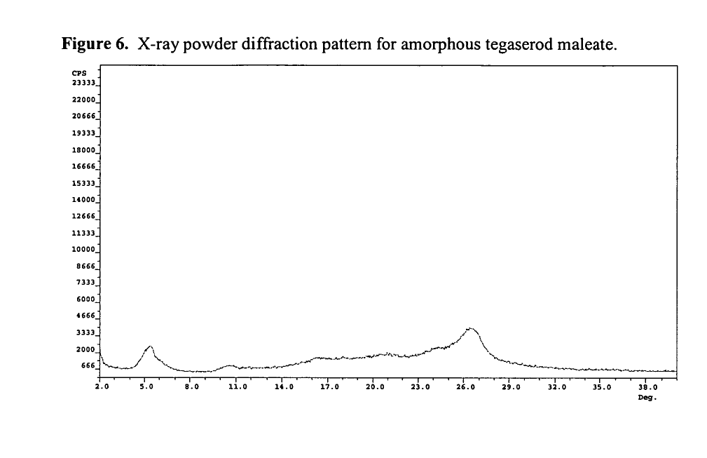

FIG. 7 illustrates an X-ray powder diffraction pattern for purely amorphous tegaserod maleate according to example 6.

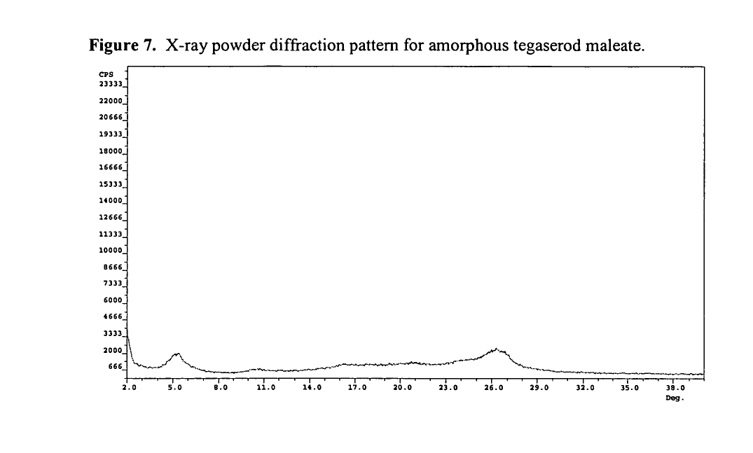

### DETAILED DESCRIPTION OF THE INVENTION

The degree of crystalinity of the portion of the crystalline material is established using powder X-ray diffraction. The integrated peak intensity of the crystalline peaks divided by the overall integrated area of the pattern is used to deduce the percent of the crystalline portion. Crystalline peaks produced by an X-ray diffraction measurement, are characterized by having a half-value width below 2 degrees.

Amorphous solids, in contrast to crystalline forms, do not possess a distinguishable crystal lattice and do not have an orderly arrangement of structural units. Amorphous forms are generally more soluble, and thus they are desirable for pharmaceutical purposes because the bioavailability of amorphous compounds may be greater than their crystalline counterparts.

In one aspect, the invention encompasses amorphous tegaserod maleate. Preferably, the amorphous tegaserod maleate contains less than about 20% crystalline tegaserod maleate by weight, more preferably less than about 10% by weight, and even more preferably less than about 5% by weight.

Preferably, the amorphous form is free of detectable tegaserod maleate Form A crystalline peaks.

When the amorphous tegaserod maleate has less than about 1% crystalline tegaserod maleate by weight, the tegaserod maleate is purely amorphous.

In another aspect, the invention encompasses purely amorphous tegaserod maleate.

Preferably, the purely amorphous form is free of detectable tegaserod maleate Form A crystalline peaks.

The invention encompasses processes for preparing amorphous tegaserod maleate by spray drying.

The term “spray drying” broadly refers to processes involving breaking up liquid mixtures into small droplets (atomization) and rapidly removing solvent from the mixture. In a typical spray drying apparatus, there is a strong driving force for evaporation of solvent from the droplets, which may be provided by providing a drying gas. Spray drying processes and equipment are described in Perry's Chemical Engineer's Handbook, pgs. 20-54 to 20-57 (Sixth Edition 1984).

By way of non-limiting example only, the typical spray drying apparatus comprises a drying chamber, atomizing means for atomizing a solvent-containing feed into the drying chamber, a source of drying gas that flows into the drying chamber to remove solvent from the atomized-solvent-containing feed, an outlet for the products of drying, and product collection means located downstream of the drying chamber. Examples of such apparatuses include Niro Models PSD-1, PSD-2 and PSD-4 (Niro A/S, Soeborg, Denmark). Typically, the product collection means includes a cyclone connected to the drying apparatus. In the cyclone, the particles produced during spray drying are separated from the drying gas and evaporated solvent, allowing the particles to be collected. A filter may also be used to separate and collect the particles produced by spray drying. Spray-drying may be performed in a conventional manner in the processes of the present invention (see, e.g., Remington: The Science and Practice of Pharmacy, 19th ed.; vol. 11, pg. 1627, herein incorporated by reference). The drying gas used in the invention may be any suitable gas, although inert gases such as nitrogen, nitrogen-enriched air, and argon are preferred. Nitrogen gas is a particularly preferred drying gas for use in the process of the invention. The ziprasidone mesylate product produced by spray-drying may be recovered by techniques commonly used in the art, such as using a cyclone or a filter.

The process of the present invention comprises: providing a solution of tegaserod maleate in at least one organic solvent and spray drying the solution to obtain amorphous tegaserod maleate.

The tegaserod maleate in the solution may be any crystalline or other form of tegaserod maleate, including various solvates and hydrates, as long as amorphous tegaserod maleate is produced during the spray drying process of the invention. When in solution, the crystalline form of the starting material does not affect the final result since the original form is lost.

Suitable organic solvents include at least one of a C₁-C₈ alcohol, a C₃-C₈ ketone, a C₂-C₈ ethers, a C₃-C₈ esers an aliphatic nitrile, dioxane, butyl lactate, ethyl lactate, cellosolve, tetrahydrofuran (THF), Dimethylamine (DMA), Dimethylformamide (DMF), Dimethyl Sulfoxide (DMSO), methylpyrrolidone, and ethylene glycol. Preferred alcohols include methanol, ethanol, and propanol. Preferred ketones include acetone and methyl ethyl ketone. Preferred nitriles include acetonitrile. The more preferred solvent is methanol. The amount of the solvent used is at least about 20 volumes of the tegaserod maleate.

Tegaserod maleate can be present in any amount that will produce the amorphous form upon spray drying. Preferably, the tegaserod maleate is present in an amount of about 1% to about 30% by weight of the organic solvent, more preferably about 1% to about 20% by weight, more preferably about 1% to about 10% by weight, and most preferably about 2% to about 7% by weight. One skilled in the art would understand that depending on the choice of solvent, the amount of tegaserod maleate used may be varied. For example, when the solvent is methanol, a preferred range may be from about 2% to about 7% of tegaserod maleate by weight of methanol.

The solution may be heated to dissolve the tegaserod maleate. The temperature suitable for dissolving tegaserod maleate depends on the organic solvent used and the amount of tegaserod maleate in the solution. Typically, the solution is heated at a temperature of at least about 30° C. to about reflux. Preferably, the solution is heated at about 40° C. to about 65° C., and more preferably at about 40° C. to about 50° C. The solution may be prepared at other suitable temperatures as long as the tegaserod maleate is sufficiently dissolved. Increasing the amount of tegaserod maleate would generally require the use of higher temperatures. Routine experimentation will provide the approximate range of suitable temperatures for a given organic solvent and amount of tegaserod maleate.

After the tegaserod maleate is dissolved, the solution may optionally be cooled to about room temperature, or about 25° C.

The gas inlet temperature during spray drying is about 10° C. to about 220° C. More preferably, the gas inlet temperature is about 25° C. to about 200° C., and most preferably about 25° C. to about 150° C. An “inlet temperature” is the temperature at which the solution enters the spray dryer.

The outlet temperature is preferably below the inlet temperature, more preferably, the outlet temperature is from about 5° C. to about 100° C. Even more preferably, the outlet product is from about 5° C. to about 60° C., and most preferably about 5° C. to about 45° C. An “outlet temperature” is the temperature at which the gas exits the spray dryer.

Inlet or outlet temperatures may be varied, if necessary, depending on the equipment, gas, or other experimental parameters. For example, it is known that the outlet temperature may depend on parameters such as aspirator rate, air humidity, inlet temperature, spray air flow, feed rate or concentration.

The solution of tegaserod maleate that is spray dried may be prepared from tegaserod acetate, tegaserod hemi-maleate hemihydrate, or sesqui-tegaserod maleate hemihydrate. The process comprises combining tegaserod acetate, tegaserod hemi-maleate hemihydrate, or sesqui-tegaserod maleate hemihydrate and maleic acid in at least one organic solvent, and spray drying the solution to obtain amorphous tegaserod maleate. Preferably, the organic solvent, as well as the spray drying conditions are as described above.

Tegaserod acetate may be prepared according to the process disclosed in PCT publication no. WO 2005/058819.

Tegaserod hemi-maleate hemi hydrate may be prepared according to any one of the processes disclosed in PCT publication no. WO 2005/058819 or WO 2006/002212.

Sesqui-tegaserod maleate hemihydrate may be prepared according to U.S. provisional application No. 60/760,306.

Amorphous tegaserod maleate may be analyzed to determine the amorphous nature of the product. The X-ray powder diffraction pattern of amorphous tegaserod maleate would show no peaks characteristic of crystal forms of tegaserod maleate, thus demonstrating the amorphous nature of the product. The presence of peaks would indicate presence of crystalline tegaserod maleate. When there are peaks in an XRD pattern, the area under the peaks pattern may be combined to determine the total amount of crystalline material.

Amorphous or purely amorphous tegaserod maleate prepared according to the invention may be formulated into pharmaceutical compositions and dosage forms according to methods known in the art and used for the treatment of irritable bowel syndrome.

In another embodiment, the present invention provides a process for preparing amorphous tegaserod maleate comprising: providing a solution of tegaserod maleate and organic solvent and fast-removing the solvent under reduced pressure.

As used herein, the term “reduced pressure” refers to a pressure below 760 mmHg or 1 atmosphere.

Preferably, the solvent is removed under vacuum.

Preferably, the organic solvent is selected from the group consisting of: C₁ to C₄ alcohol, C₃ to C₇ ketone, C₃ to C₇ ester, C₅ to C₇ straight or cyclic saturated hydrocarbon and C₂ to C₈ ethers, or mixtures thereof. More preferably, the organic solvent is selected from the group consisting of: methanol, ethanol, acetone, ethylacetate, heptane, hexane, diethylether methyl isobutylether, or mixtures thereof. Most preferably, the organic solvent is methanol.

The concentration, solvent type, temperature, vacuum, feeding rate are set to such a combination where the tegaserod maleate, coming from the inlet, such as a nozzle, precipitates instantly. Otherwise crystalline material can also form. The process may be carried out at a temperature below about 100° C., a reduced pressure and a concentrated solution of the tegaserod maleate in a solvent, preferably having a concentration of more than about 20% m/m, and/or concentrated to the point of saturation (solution in equilibrium with a solid solute), and a flow rate of about 10 to about 50 cm³/hour/inlet. These combinations should allow for evaporation of the solvent at the given conditions, i.e., below the vapor pressure of the solvent.

The last step of the tegaserod maleate isolation process is preferably a concentration in a solvent where the tegaserod maleate is dissolved. This concentrated solution, with preferably more than about 20 m/m %, more preferably about 20 to about 80 m/m %, more preferably about 60% to about 75%, and/or a solution concentrated to the point of saturation, is fed into a reduced pressure chamber, at a temperature of less than about 100° C., through preferably a sort of nozzles (inlets). The feeding may be carried out by a pump, pressure from another tank, vacuum in the drying chamber or pressure from a syringe device. A chamber may be any reactor, flask, container capable of maintaining the desirable process conditions such as reduced pressure.

In the process of the present invention, the solution is added dropwise or continuously to the drying chamber. One skilled in the art would appreciate that the speed of the addition of the solution will depend on the solvent used, the viscosity of the mixture, and the height of the chamber. Rate of flow of the solution, if delivered through a nozzle, is preferably in the range of about 10 to about 50 cm³/hour/nozzle (inlet), depending on the concentration, pressure, temperature, properties of the solvent and the tegaserod maleate.

The drop of solution explodes (like a popcorn kernel popping) instantaneously in the chamber. This solidification is spontaneous, and does not require further actions such as stirring, and occurs as the solution comes out of the nozzle (inlet) into the drying chamber. This instant evaporation allows for obtaining a phase change (solidification) before the solution contacts the bottom of an industrial sized chamber when fed from the top. A small industrial size chamber has a height of about 0.5 to about 1 meter. It is possible to feed the solution from the side or bottom of the chamber as well.

When the solution reaches the drying chamber, the solvent instantly evaporates, while the dissolved tegaserod maleate precipitates as a sponge (a solid foam) or even possibly as a solid.

Number of inlets for the nozzles in the drying chamber depends on the capacity of vacuum. Vapor removal from the drying chamber can be accelerated by a small leak of an inert gas, preferably nitrogen. Drying equipment preferably contains a stirrer, which is suitable to break the solid, forming a powder.

After breaking the solid, tegaserod maleate drying can be continued under reduced pressure, preferably with stirring until the residual solvent concentration reduces to the required FDA level. The solvent level depends on the type of solvent but is preferably no more than about 5000 ppm, more preferably no more than about 4000 ppm, and most preferably no more than about 3000 ppm. The drying of the powder after the stirring is preferably carried out under reduced pressure (below 1 atm), more preferably below about 100 mmHg, most preferably below about 50 mmHg. The temperature is preferably about 30° C. to about 50° C., more preferably about 35° C. to about 45° C. The drying is preferably carried out for about 1 hour to about 10 hour.

The powder can be discharged from the dryer by conventional way, for example via an outlet of a chamber located at the bottom of the chamber, while the stirrer is rotating. A valve may be opened to discharge the powder, and additional force in addition to gravitational force may be used to accelerate the discharge.

The process of the present invention is preferably carried out with a feeding system having a distributor of preferably less than about 3 mm diameter syringe/nozzle, more preferably less than about 2 mm, continuous feeding of tegaserod maleate solution, working pressure of preferably less than about 760 mmHg, more preferably less than about 100 mmHg, more preferably less than about 50 mmHg, most preferably less than about 20 mmHg, working temperature of less than about 100° C., preferably about 20° C. to about 80° C., more preferably about 25° C. to about 45° C., optional inert gas flow (such as N₂), and a drying chamber with stirrer and a discharge device. While drop-wise addition is possible, scaling up is easier with a syringe and continuous feeding.

Pharmaceutical compositions containing amorphous tegaserod maleate may optionally contain a mixture of other form(s) of tegaserod maleate. In addition to the active ingredient(s), the pharmaceutical formulations may contain one or more excipients. Excipients are added to the formulation for a variety of purposes.

Pharmaceutical compositions may be prepared as medicaments to be administered orally, parenterally, rectally, transdermally, bucally, or nasally. Suitable forms for oral administration include tablets, compressed or coated pills, dragees, sachets, hard or gelatin capsules, sub-lingual tablets, syrups and suspensions. Suitable forms of parenteral administration include an aqueous or non-aqueous solution or emulsion, while for rectal administration suitable forms for administration include suppositories with hydrophilic or hydrophobic vehicle. For topical administration the invention provides suitable transdermal delivery systems known in the art, and for nasal delivery there are provided suitable aerosol delivery systems known in the art.

Selection of excipients and the amounts to use may be readily determined by the formulation scientist based upon experience and consideration of standard procedures and reference works in the field. For example, diluents increase the bulk of a solid pharmaceutical composition, and may make a pharmaceutical dosage form containing the composition easier for the patient and care giver to handle. Diluents for solid compositions include, for example, microcrystalline cellulose (e.g. Avicel®), microfine cellulose, lactose, starch, pregelitinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit), potassium chloride, powdered cellulose, sodium chloride, sorbitol and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®, guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Glidants can be added to improve the flowability of a non-compacted solid composition and to improve the accuracy of dosing. Excipients that may function as glidants include colloidal silicon dixoide, magnesium trisilicate, powdered cellulose, starch, talc and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol, and tartaric acid.

Solid and liquid compositions may also be dyed using any pharmaceutically acceptable colorant to improve their appearance and/or facilitate patient identification of the product and unit dosage level.

In liquid pharmaceutical compositions, the active ingredient and any other solid excipients are suspended in a liquid carrier such as water, vegetable oil, alcohol, polyethylene glycol, propylene glycol or glycerin.

Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Liquid pharmaceutical compositions of the present invention may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar may be added to improve the taste.

Preservatives and chelating agents such as alcohol, sodium benzoate, butylated hydroxy toluene, butylated hydroxyanisole and ethylenediamine tetraacetic acid may be added at levels safe for ingestion to improve storage stability.

According to the present invention, a liquid composition may also contain a buffer such as gluconic acid, lactic acid, citric acid or acetic acid, sodium gluconate, sodium lactate, sodium citrate or sodium acetate.

Selection of excipients and the amounts used may be readily determined by the formulation scientist based upon experience and consideration of standard procedures and reference works in the field.

The solid compositions of the present invention include powders, granulates, aggregates and compacted compositions. The dosages include dosages suitable for oral, buccal, rectal, parenteral (including subcutaneous, intramuscular, and intravenous), inhalant and ophthalmic administration. Although the most suitable administration in any given case will depend on the nature and severity of the condition being treated, the most preferred route of the present invention is oral. The dosages may be conveniently presented in unit dosage form and prepared by any of the methods well-known in the pharmaceutical arts.

Dosage forms include solid dosage forms like tablets, powders, capsules, suppositories, sachets, troches and lozenges, as well as liquid syrups, suspensions and elixirs.

The dosage form of the present invention may be a capsule containing the composition, preferably a powdered or granulated solid composition of the invention, within either a hard or soft shell. The shell may be made from gelatin and optionally contain a plasticizer such as glycerin and sorbitol, and an opacifying agent or colorant.

The active ingredient and excipients may be formulated into compositions and dosage forms according to methods known in the art.

A composition for tableting or capsule filling may be prepared by wet granulation. In wet granulation, some or all of the active ingredients and excipients in powder form are blended and then further mixed in the presence of a liquid, typically water, that causes the powders to clump into granules. The granulate is screened and/or milled, dried and then screened and/or milled to the desired particle size. The granulate may then be tableted, or other excipients may be added prior to tableting, such as a glidant and/or a lubricant.

A tableting composition may be prepared conventionally by dry blending. For example, the blended composition of the actives and excipients may be compacted into a slug or a sheet and then comminuted into compacted granules. The compacted granules may subsequently be compressed into a tablet.

As an alternative to dry granulation, a blended composition may be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling of the present invention may comprise any of the aforementioned blends and granulates that were described with reference to tableting, however, they are not subjected to a final tableting step.

The pharmaceutical compositions of the present invention, used to treat irritable bowel syndrome in a mammal such as a human, are preferably in the form of a coated tablet, and are administered on an empty stomach twice a day, for a period of about 4 to about 6 weeks. Additional administration may occur if the patient responds positively to the treatment.

Having thus described the invention with reference to particular preferred embodiments and illustrative examples, those in the art may appreciate modifications to the invention as described and illustrated that do not depart from the spirit and scope of the invention as disclosed in the specification. The following examples are set forth to aid in understanding the invention but are not intended to, and should not be construed to, limit its scope in any way. The examples do not include detailed descriptions of conventional methods. Such methods are well known to those of ordinary skill in the art and are described in numerous publications. Polymorphism in Pharmaceutical Solids, Drugs and the Pharmaceutical Sciences, Volume 95 may be used as a guidance.

### EXAMPLES

Instruments

Spray drying was performed using a Buchi mini spray dryer B-290 using a standard nozzle 0.7 mm in diameter with a nozzle cap of 1.4 or 1.5 mm.

X-Ray powder diffraction (XRD) data is obtained using a SCINTAG powder X-Ray diffractometer model X'TRA equipped with a solid state detector. Copper radiation of 1.5418 Å is used. A round aluminum sample holder with zero background is used. All peak positions are within ±0.2 degrees two theta.

### Example 1

### Preparation of Amorphous Tegaserod Maleate

10 g of tegaserod maleate was dissolved in 264 g methanol at 50° C. The spray solution was pumped into the spray dryer and nitrogen, at an inlet temperature of 100° C., was provided as a drying gas. The evaporated solvent and nitrogen exited the spray drier at 65-70° C. The obtained sample was analyzed by XRD and determined to be amorphous form with approximately 15% crystalline tegaserod maleate by weight.

### Example 2

### Preparation of Amorphous Tegaserod Maleate

10 g of tegaserod maleate was dissolved in 480 g methanol. The spray solution was pumped into the spray dryer at 60° C. Nitrogen, at an inlet temperature of 100° C., was provided as a drying gas. The evaporated solvent and nitrogen exited the spray drier at 56-63° C. The obtained sample was analyzed by XRD and determined to be amorphous form with approximately 5% crystalline tegaserod maleate by weight.

### Example 3

### Preparation of Amorphous Tegaserod Maleate

15 g of tegaserod maleate was dissolved in 720 g methanol. The spray solution was pumped into the spray dryer at 60° C. Nitrogen, at an inlet temperature of 150° C., was provided as a drying gas. The evaporated solvent and nitrogen exited the spray drier at 94-96° C. The obtained sample was analyzed by XRD and determined to be amorphous form with approximately 20% crystalline tegaserod maleate by weight.

### Example 4

### Preparation of Purely Amorphous Tegaserod Maleate

2 g of tegaserod maleate was dissolved in 200 ml methanol at 40° C., and the solution was cooled to 25° C. The spray solution was pumped into the spray dryer Nitrogen, at an inlet temperature of 25° C., was provided as a drying gas. The evaporated solvent and nitrogen exited the spray drier at 15-20° C. The obtained sample was analyzed by XRD and determined to be purely amorphous form.

### Example 5

### Preparation of Purely Amorphous Tegaserod Maleate

5 g of tegaserod maleate was dissolved in 175 ml methanol at reflux temperature, and the spray solution was pumped into the spray dryer. Nitrogen, at an inlet temperature of 100° C., was provided as a drying gas. The evaporated solvent and nitrogen exited the spray drier at 58-61° C. The obtained sample was analyzed by XRD and determined to be purely amorphous form.

### Example 6

### Preparation of Purely Amorphous Tegaserod Maleate

5 g of tegaserod maleate was dissolved in 250 ml methanol at reflux temperature, and the spray solution was pumped into the spray dryer. Nitrogen, at an inlet temperature of 100° C., was provided as a drying gas. The evaporated solvent and nitrogen exited the spray drier at 60-61° C. The obtained sample was analyzed by XRD and determined to be purely amorphous form.

### Example 7

### Preparation of Amorphous Tegaserod Maleate from Tegaserod Acetate

8.66 g of tegaserod acetate and 2.8 g of maleic acid are heated to reflux in 480 g methanol. The spray solution is pumped into the spray dryer at 60° C.; the nitrogen was at an inlet temperature of 100° C. The evaporated solvent and nitrogen exit the spray drier at 56-63° C. The obtained sample is analyzed by XRD.

### Example 8

### Preparation of Amorphous Tegaserod Maleate from Tegaserod Hemi Maleate Hemi Hydrate

17.65 g of tegaserod hemi maleate hemi hydrate and 2.8 g of maleic acid are heated to reflux in 480 g methanol. The spray solution is pumped into the spray dryer at 60° C.; the nitrogen was at an inlet temperature of 100° C. The evaporated solvent and nitrogen exit the spray drier at 56-63° C. The obtained sample is analyzed by XRD.

### Example 9

### Preparation of Amorphous Tegaserod Maleate from Sesqui-Tegaserod Maleate Hemi Hydrate

28.08 g of sesqui-tegaserod maleate hemi hydrate and 2.8 g of maleic acid are heated to reflux in 480 g methanol. The spray solution is pumped into the spray dryer at 60° C.; the nitrogen was at an inlet temperature of 100° C. The evaporated solvent and nitrogen exit the spray drier at 56-63° C. The obtained sample is analyzed by XRD.

### Example 10

### Preparation of Amorphous Tegaserod Maleate

Tegaserod maleate is dissolved in methanol (50 Volumes) at elevated temp. introduced-injected and evacuated (5-20 mbar) at jacket temperature 40° C., through 8 nozzles to dryness. After feeding, the product is broken by a mechanic stirrer and dried under vacuum (5-20 mbar) at 35° C. for 8 hours. The final product is dried under vacuum (5-20 mbar) for 2 hours at 50° C.

1. Purely amorphous tegaserod maleate.

2. The purely amorphous tegaserod maleate of claim 1 having an X-ray powder diffraction pattern free of detectable tegaserod maleate Form A.

3. Amorphous tegaserod maleate.

4. The amorphous tegaserod maleate of claim 3 containing less than about 20% crystalline tegaserod maleate by weight.

5. The amorphous tegaserod maleate of claim 4 containing less than about 10% crystalline tegaserod maleate by weight.

6. The amorphous tegaserod maleate of claim 5 containing less than about 5% crystalline tegaserod maleate by weight.

7. The amorphous tegaserod maleate of claim 6 having an X-ray powder diffraction pattern free of detectable tegaserod maleate Form A.

8. A process for preparing the amorphous tegaserod maleate of claim 3 comprising:
a. providing a solution of tegaserod maleate in at least one organic solvent; and
b. spray drying the solution to obtain amorphous tegaserod maleate.

9. A process for preparing the amorphous tegaserod maleate of claim 3 comprising:
a. combining tegaserod acetate, tegaserod hemi-maleate hemihydrate, or sesqui-tegaserod maleate hydrate with maleic acid in at least one organic solvent to obtain a solution; and
b. spray drying the solution to obtain amorphous tegaserod maleate.

10. The process of any of claims 8 or 9, wherein the organic solvent is selected from the group consisting of: a C₁-C₈ alcohol, a C₃-C₈ ketone, a C₂-C₈ ethers, a C₃-C₈ esters, an aliphatic nitrile, dioxane, butyl lactate, ethyl lactate, cellosolve, tetrahydrofuran (THF), Dimethylacetamide (DMA), Dimethylformamide (DMF), Dimethyl Sulfoxide (DMSO), methylpyrrolidone and ethylene glycol.

11. The process of claim 10, wherein the organic solvent is selected from the group consisting of: methanol, ethanol, propanol, acetone, methyl ethyl ketone and acetonitrile.

12. The process of claim 11, wherein the organic solvent is methanol.

13. (canceled)

14. The process of any of claims 8 or 9, wherein the tegaserod maleate is present in an amount of about 1% to about 30% by weight of the organic solvent.

15. The process of claim 14, wherein the tegaserod maleate is present in an amount of about 1% to about 20% by weight of the organic solvent.

16. The process of claim 15, wherein the tegaserod maleate is present in an amount of about 1% to about 10% by weight of the organic solvent.

17. The process of claim 16, wherein the tegaserod maleate is present in an amount of about 2% to about 7% by weight of the organic solvent.

18. The process of any of claims 8 or 9, wherein the solution is heated at a temperature of at least about 30° C. to about reflux.

19. The process of claim 18, wherein the solution is heated at about 40° C. to about 65° C.

20. The process of claim 19, wherein the solution is heated at about 40° C. to about 50° C.

21. The process of claim 18, wherein prior to step b), the solution is cooled to about room temperature.

22. The process of any of claims 8 or 9, wherein step b), is performed using an inlet temperature of about 10° C. to about 220° C.

23. The process of claim 22, wherein the inlet temperature is from about 25° C. to about 200° C.

24. The process of claim 23, wherein the inlet temperature is from about 25° C. to about 150° C.

25. The process of any of claims 8 or 9, wherein step b), is performed using an outlet temperature below the inlet temperature.

26. The process of claim 25, wherein the outlet temperature is from about 5° C. to about 100° C.

27. The process of claim 26, wherein the outlet temperature is from about 5° C. to about 60° C.

28. The process of claim 27, wherein the outlet temperature is from about 5° C. to about 45° C.

29. The process of any of claims 8 or 9, wherein a purely amorphous tegaserod maleate is obtained.

30. A pharmaceutical composition comprising the amorphous tegaserod maleate of claims 1 or 6 and at least one pharmaceutically-acceptable carrier.

31. A method of treating irritable bowl syndrome comprising administering a therapeutically effective amount of the pharmaceutical composition of any of claims 30 to a mammal in need thereof.
